(19) United States
(12) Patent Application Publication
(10) Pub. No.: US 2022/0323102 A1
(43) Pub. Date: Oct. 13, 2022

(54) SYSTEM FOR LEAD PLACEMENT VIA MARKERS AND MEASUREMENTS

(71) Applicant: Medtronic, Inc., Minneapolis, MN (US)

(72) Inventors: Elliot C. Schmidt, Minneapolis, MN (US); Ryan Phillip Lahm, Lino Lakes, MN (US); Robert C. Kowal, Excelsior, MN (US); Varun A. Bhatia, Minneapolis, MN (US)

(21) Appl. No.: 17/658,402

(22) Filed: Apr. 7, 2022

Related U.S. Application Data
(60) Provisional application No. 63/172,355, filed on Apr. 8, 2021.

(51) Int. Cl. A61B 17/34; A61N 1/05

(52) U.S. Cl. CPC A61B 17/3415; A61B 17/3468; A61N 1/05; A61B 2090/3937

(57) ABSTRACT

An implant tool system includes an implantable medical lead and a delivery catheter. The implantable medical lead is configured to be delivered through an access point on a body of a patient. The delivery catheter is configured to deliver the implantable medical lead along an implantation path in the body of the patient. A visual marker is positioned on a body of the implantable medical lead. The visual marker is positioned proximal a distal end of the implantable medical lead by a distance indicative of a length of lead to introduce into the body of the patient. The visual marker may be positioned such that introduction of the length of lead into the body of the patient provides lead slack that reduces the likelihood of one or more of dislodgment of the implantable medical lead from the implant point or damage to a tissue of the patient.

This application claims the benefit of U.S. Provisional Application Ser. No. 63/172,355, filed Apr. 8, 2021, the entire content of which is incorporated herein by reference.

### TECHNICAL FIELD

This disclosure relates generally to implant tool systems.

### BACKGROUND

Some types of implantable medical devices (IMDs), such as cardiac pacemakers or implantable cardioverter defibrillators systems, may be used to provide cardiac sensing and therapy for a patient via one or more electrodes. Some IMDs include one or more implantable medical electrical leads that include one or more electrodes. The leads may be configured such that the electrodes may, as examples, be implanted within the heart (e.g., transvenous leads) or outside of the heart and vasculature (e.g., extravascular leads). Once the leads are implanted, tines or other fixation elements attached to various locations of the leads may be deployed to prevent the leads from shifting or moving.

### SUMMARY

In an example, an implant tool system comprises: an implantable medical lead configured to be delivered through an access point on a body of a patient; a delivery catheter configured to deliver the implantable medical lead along an implantation path in the body of the patient; and a visual marker positioned: on a body of the implantable medical lead, and proximal a distal end of the implantable medical lead by a distance indicative of a length of lead to introduce into the body of the patient.

In an example, a method of using an implant tool system for delivering an implantable medical lead through an access point on a body of a patient comprises: inserting, into the body of the patient, an introducer; advancing the introducer along an implantation path in the body of the patient until a distal end of the introducer abuts an implant point; positioning, based on the length of the introducer inserted into the body of the patient when the distal end of the introducer abuts the implant point, a visual marker on a body of the implantable medical lead, wherein the visual marker is positioned proximal a distal end of the implantable medical lead by a distance indicative of a length of lead to introduce into the body of the patient.

In an example, an implant tool system comprises: an implantable medical lead configured to be delivered through an access point on a body of a patient; a delivery catheter configured to deliver the implantable medical lead along an implantation path in the body of the patient; a first visual marker positioned: on a body of the implantable medical lead, and proximal a distal end of the implantable medical lead by a first distance indicative of a minimum length of lead to introduce into the body of a patient such that the distal end of the implantable medical lead abuts an implant point; and a second visual marker positioned: on the body of the implantable medical lead and proximal the distal end of the implantable medical lead by a second lead distance indicative of an additional length of lead to introduce into the body of the patient, or on the body of the delivery catheter and proximal a distal end of the delivery catheter by a second catheter distance indicative of the additional length of lead to introduce into the body of the patient.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1A is a conceptual drawing illustrating an example implant tool system including visual markers indicating lengths of lead to introduce into a patient's body.

FIG. 1B is a conceptual drawing illustrating an example introducer and an example implantable medical lead.

FIG. 2A is a conceptual drawing illustrating an example implant tool system including visual markers indicating lengths of lead to introduce into a patient's body and a length of a delivery catheter to withdraw from the patient's body.

FIG. 2B is a conceptual drawing illustrating an example implant tool system including visual markers indicating lengths of lead to introduce into a patient's body and a length of a delivery catheter to withdraw from the patient's body.

FIG. 3 is a conceptual drawing illustrating an example implantable medical lead including visible indicia representing a series of measurement gradations.

FIG. 4 is a flow diagram of an example technique for delivering an appropriate length of lead into a patient's body.

FIG. 5 is a flow diagram of an example technique for delivering an appropriate length of lead into a patient's body.

### DETAILED DESCRIPTION

A clinician may be required to determine a length of implantable medical lead to introduce into a body of a patient in order to efficaciously position and secure the implantable medical lead. If a clinician introduces a disadvantageous (e.g., insufficient or excessive) length of implantable medical lead into the patient's body, the implantable medical lead may be liable to dislodge from tissue of an implant point (e.g., tissue of a heart of a patient), damage the tissue, impair sensing of electrical signals from the tissue, and/or impair delivery of therapy.

This disclosure describes example implant tool systems that help a clinician or other user to determine a length of implantable medical lead to introduce into the patient's body. Such implant tool systems may include an implantable medical lead, a delivery catheter, and a visual marker. The implantable medical lead may be configured to be delivered through an access point (e.g., a venous entry site) on a body of a patient. For example, the implantable medical lead may translate within a lumen of the delivery catheter toward a distal end of the delivery catheter and, in turn, the implant point. The delivery catheter may be configured to deliver the implantable medical lead along an implantation path (e.g., a vasculature of the patient) in the patient's body. The visual marker may be configured to be visible to the clinician with the naked eye or otherwise without the use of fluoroscopic or other visualization techniques.

By using the visual marker of the implant tool system, a clinician may advantageously determine a length of implantable medical lead to introduce into the body's patient. For example, the visual marker may be positioned on a body of the implantable medical lead. Additionally, the visual marker may be positioned proximal a distal end of the implantable medical lead by a distance indicative of a length of lead to introduce into the body of the patient. In some examples, the visual marker may be printed, sprayed, written, or otherwise impressed upon a body of the implantable medical lead. In other examples, the visual marker may be a sheath, band, or other member that may be reversibly secured to the implantable medical lead's body. A visual marker that is reversibly secured to the implantable medical lead's body may be configured to translate (e.g., move, slide, etc.) along the implantable medical lead's body in response to an external force (e.g., applied by a clinician). Such a visual marker may fit tightly enough to prevent inadvertent movement of the visual marker relative to the implantable medical lead's body.

To use the implant tool system, a physician may insert the implantable medical lead into a patient's body via a delivery catheter. For example, the implantable medical lead may pass through a proximal end of the delivery catheter at an access point (e.g., a venous entry site) toward an implant point. The implantable medical lead may be translated into the delivery catheter until a visual marker on the body of the implantable medical lead is aligned with a reference point, such as surface of the patient's body defining the access point or the proximal end of the delivery catheter. This alignment may indicate that an adequate length of lead has been introduced into the body of the patient. Introducing this indicated length of lead may advantageously reduce the likelihood of dislodging the implantable medical lead from an implant point (e.g., tissue of a heart of a patient), damaging tissue of the patient's heart, impairing sensing, and/or impairing delivery of therapy.

The example implant tool systems described herein enable a clinician to visually identify whether an advantageous length of implantable medical lead has been introduced into the patient's body. Thus, rather than guess a length of implantable medical lead to introduce into the patient's body or use fluoroscopic or other visualization techniques (that may adversely affect a patient) to determine a length of lead introduced into the patient's body, a clinician may use the techniques described herein to advantageously determine a length of implantable medical lead to introduce into the patient's body, potentially increasing the safety and efficacy of implantation procedures.

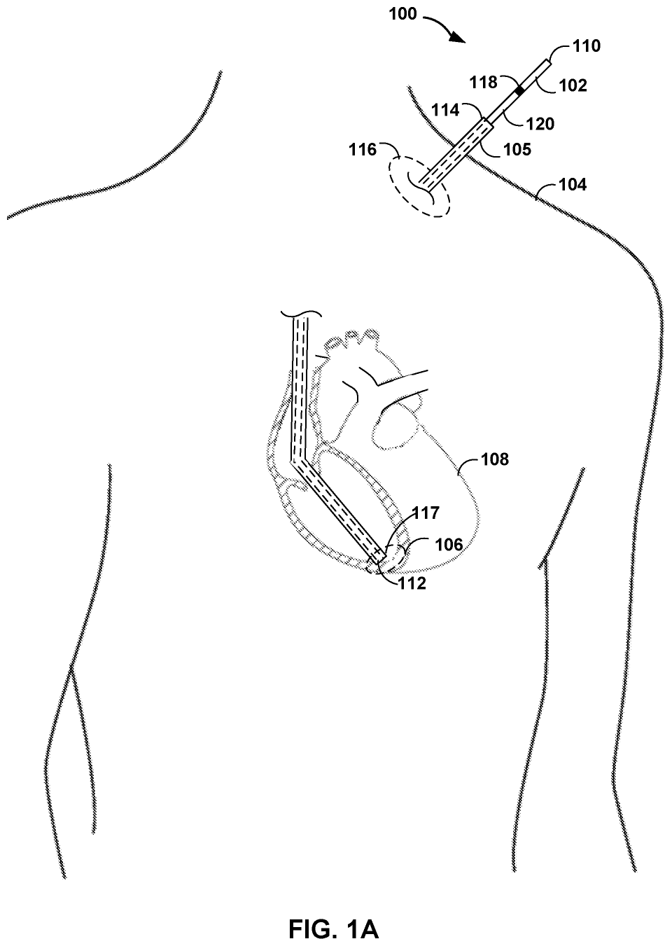

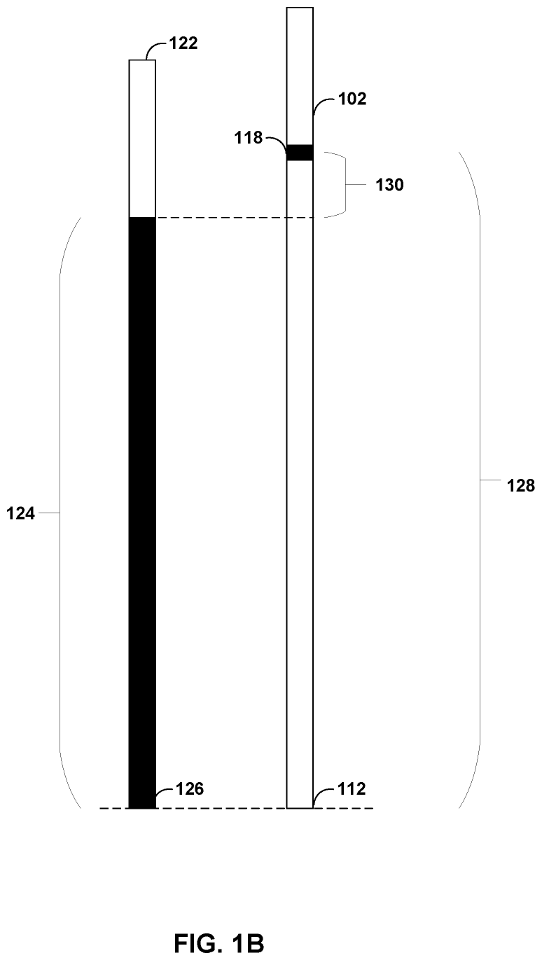

FIG. 1A is a conceptual diagram of an example implant tool system 100 (“system 100”) for implanting an implantable medical lead 102 (“lead 102”) within a patient 104. In the example illustrated in FIG. 1A, system 100 includes lead 102 that provides pacing therapy and a delivery catheter 105 (“catheter 105”). However, some of the components of system 100 described herein, such as one or more visual markers, may be utilized with other types of implant tool systems, such as implant tool systems for delivering IMDs configured to deliver electrical therapy other than cardiac electric therapy. Thus, although system 100 is described herein in the context of cardiac electrical therapy, one or more aspects of system 100 may be adapted to other types of implant tool systems. In addition, it should be noted that system 100 may not be limited to treatment of a human patient. System 100 may be implemented in non-human patients, such as primates, canines, equines, pigs, ovines, bovines, felines, and/or the like. These non-human patients may undergo clinical or research therapies that may benefit from the subject matter of this disclosure.

In general, a clinician may insert lead 102 into and through a patient's vasculature to an implant point 106 (e.g., tissue of a heart 108 of patient 104) where a medical procedure may be undertaken. Lead 102 may have a proximal end 110 (“lead proximal end 110”) and a distal end 112 (“lead distal end 112”). Lead distal end 112 may be inserted into a proximal end 114 of catheter 105 (“delivery catheter proximal end 114”) exposed at an access point 116. That is, a portion of delivery catheter 105 may be intracorporeal, while a remaining portion of catheter 105 may be extracorporeal. As shown in FIG. 1A, catheter 105 may be positioned within the patient's body such that a distal end 117 of catheter 105 (“delivery catheter distal end 117”) abuts implant point 106.

In accordance with techniques of this disclosure, one or more of lead 102 or delivery catheter 105 may include one or more visual markers that a clinician may visually reference and potentially feel during an implantation procedure to help the clinician determine a length of lead 102 to introduce into the patient's body. For example, system 100 may include a visual marker 118 positioned on a body 120 of lead 102 (“lead body 120”). In some examples, visual marker 118 may be integrated into lead 102. For example, visual marker 118 may be printed, sprayed, written, or otherwise impressed upon lead body 120. Thus, visual marker 118 may be integrated into lead 102. Visual marker 118 may not materially change the overall diameter of lead 102 although visual marker 118 may optionally have a substantial width, depth, and/or density sufficient to make visual marker 118 visually perceptible. Furthermore, although shown as a band in FIG. 1, visual marker 118 may possess any shape or have any appearance. Thus, other configurations of visual marker 118, such as a marker in the shape of a circle (e.g., a dot), a line, a cross, and/or the like, are contemplated.

In some examples, visual marker 118 may be a sheath (e.g., a plastic sheath with markings or other visible qualities), a collar, a tie, or other member that may be reversibly secured to lead 102. For instance, visual marker 118 may be attached, clipped, tied, tightened, or otherwise coupled to lead body 120. Visual marker 118 may be repositioned on lead 102 (e.g., in response to an initial position of visual marker 118 being incorrect). In examples, visual marker 118 may be movable relative to lead 102. For example, visual marker 118 may be configured to slide on lead body 120 in response to an external force (e.g., applied by a clinician). In this way, a clinician may slidably adjust the position of visual marker 118 on lead 102 such that the position of visual marker 118 indicates the length of lead 102 to introduce into patient's body. Visual marker 118 may fit tightly enough to prevent inadvertent movement of first visual marker 118 relative to lead 102.

Visual marker 118 may be positioned on lead body 120 in accordance with techniques of this disclosure to help a clinician determine a length of lead 102 to introduce into the patient's body. For example, visual marker 118 may be positioned such that alignment of visual marker 118 with a reference point, such as the surface of the patient's body defining access point 116, indicates that an adequate or desired length of lead 102 has been introduced into the patient's body. Introducing such a length may provide lead slack (i.e., a length of lead introduced into the body of a patient in excess of the minimum length of lead required for a distal end of lead 102 to abut implant point 106) that reduces the likelihood of one or more of dislodgment of the implantable medical lead from the implant point or damage to a tissue of patient heart. It should be understood that features other than access point 116 may be the reference point, such as delivery catheter proximal end 114, another component of system 100, and/or the like.

In some examples, the position of visual marker 118 on lead body 120 may be based on a length of an introducer (not shown in FIG. 1A), such as a guidewire or stylet, inserted into the patient's body. For instance, prior to introduction of lead 102, a clinician may insert the introducer through access point 116 and advance the introducer along the implantation path until a distal end of the introducer abuts implant point 106. The length of the introducer inserted into the patient's body (e.g., from access point 116 to implant point 106) may indicate a minimum length of lead 102 required for lead 102 to reach implant point 106. Introducing the minimum length of lead 102 may enable lead distal end 112 to reach implant point 106, but implantation and fixation of lead 102 with only the minimum length of lead 102 may cause lead 102 to be relatively taut and at risk of dislodgement.

Accordingly, based on the length of the introducer inserted into the patient's body, a clinician may position visual marker 118 on lead body 120. For example, the clinician may withdraw the introducer from the patient's body, compare the introducer to lead 102, and position visual marker 118 (e.g., a collar, a band, etc.) proximal lead distal end 112 by a length including the length of the introducer inserted into the patient's body as well as a desirable length of lead slack (described in greater detail below).

By using system 100 to determine whether an appropriate length of lead 102 has been introduced into the patient's body, a clinician may avoid or limit the use of fluoroscopic observation of lead 102. For example, the clinician may determine whether a desirable length of lead 102 has been introduced into the patient's body without using fluoroscopic observation by visually comparing a location of visual marker 118 with a location of a reference point (e.g., by seeing whether visual marker 118 and access point 116 are aligned). Thus, techniques of this disclosure may prevent dislodgement of lead 102 from tissue of implant point 106 and/or reduce the patient's exposure to fluoroscopic radiation, potentially increasing the safety of implantation procedures.

FIG. 1B is a conceptual drawing illustrating implantable medical lead 102 and an introducer 122. Introducer 122 may be configured to guide another component of system 100, such as catheter 105, along an implantation path within patient's body. For example, introducer 122 may be inserted through a lumen (e.g., a central lumen of catheter 105) and through access point 116 and advanced to implant point 106. Catheter 105 may then translate along introducer 122. In some examples, introducer 122 may be a guidewire (e.g., a thin, flexible, medical wire).

As discussed above, a length 124 of introducer 122 may indicate a minimum distance from a reference point (e.g., delivery catheter proximal end 114, access point 116, etc.) to implant point 106 along an implantation path. This minimum distance may correspond to the minimum length of lead 102 required for lead 102 to reach implant point 106 from the same reference point. Thus, in some examples, the position of visual marker 118 on lead body 120 may be based on length 124. For instance, after fully advancing introducer 122, the clinician may observe length 124 inserted. The clinician may withdraw introducer 122 and align introducer 122 and lead 102. In some examples, the clinician may align a distal end 126 of introducer 122 (“introducer distal end 126”) and lead distal end 112. The clinician may then position visual marker 118 a length 128 from lead distal end 112, where length 128 includes length 124 and a length 130 of lead slack.

Length 130 of lead slack may be based on one or more factors, such as heart size, chest volume, and implant point 106. The relationship between length 130 of lead slack may be any appropriate (mathematical) relationship. For example, length 130 of lead slack may be 10% of right ventricle (RV) length of a patient as indicated by an ultrasound image. The relationship may be organized and presented in a suitable form (e.g., a table) to help a clinician determine an appropriate position for second visual marker 238. For example, a clinician may reference a table indicating length 130 of lead slack for a given heart size, chest volume, and implant point 106. At least some of these anatomical measurements may be determined based on ultrasound images. Thus, the techniques of this disclosure may help a clinician determine a length of lead 102 that not only enables lead distal end 112 to reach implant point 106, but also reduces the likelihood of dislodging lead 102 from an implant point 106, damaging tissue (e.g., of heart 108), impairing sensing, and/or impairing delivery of therapy.

In some examples, one or more of the visual markers may be positioned on one or more devices separate from lead 102. For example, one or more visual markers may be positioned on introducer 122 used during the initial introduction of catheter 105. Introducer 122 (e.g., a guidewire) may be inserted through a lumen of catheter 105 (which in other medical contexts may provide an atraumatic guide tip at distal end 117 of delivery catheter 105) to help advance catheter 105 through the vasculature of the patient. Once distal end 126 of introducer 122 is positioned adjacent to implant point 106, a visual marker may be positioned on introducer 122 indicating the length of introducer 122 introduced into the patient's body. Once distal end 117 of catheter 105 is positioned adjacent to implant point 106, introducer 122 is removed and set aside to allow the introduction and advancement of lead 102 through catheter 105. With introducer 122 removed, introducer 122 may provide a convenient tool for measuring and marking one or more of the relative distances discussed above. For example, lead 108 may be compared to introducer 122, and visual marker 118 may be positioned on lead 102 based to some extent on the visual marker positioned on introducer 122.

In some examples, introducer 122 may include a visible marker, may be marked by the physician, or may be cut to a length (e.g., allowing the end of the introducer to serve as the visual reference) that represents the desired distance between access point 116 and proximal end 110 of lead 102 when either lead 102 is in the minimal length or taut configuration (e.g., when visual marker 118 is aligned with delivery catheter proximal end 114 at the minimal introduction distance) or when lead 102 will be in the final implanted or slacked configuration. In the latter arrangement, introducer 122 may be positioned such that introducer distal end 126 aligns with access point 116. When lead 102 is subsequently advanced further into the chamber of the heart, the final implanted or slacked configuration of lead 102 may be obtained when lead proximal end 114 is aligned with the visual marker on introducer 122 or aligned with the cut length of introducer 122.

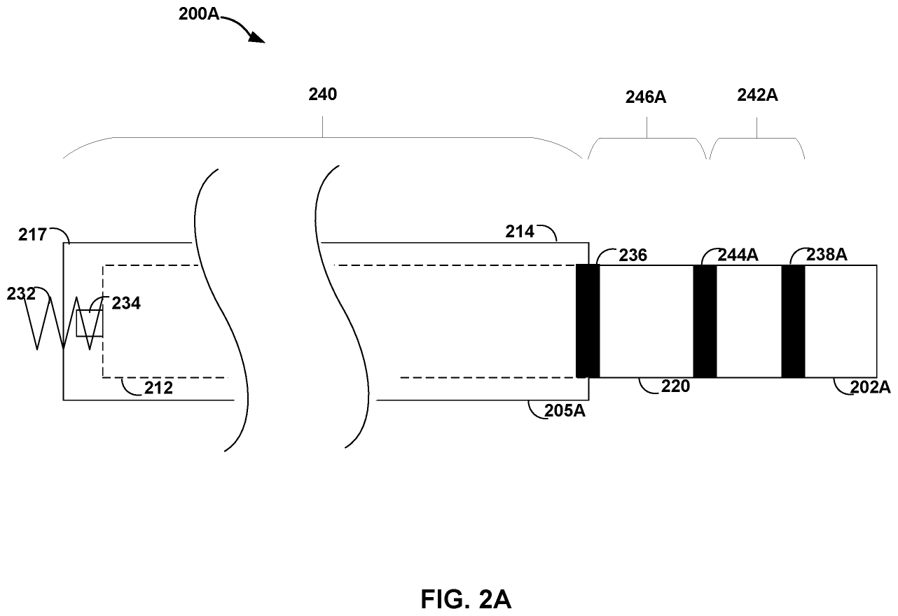

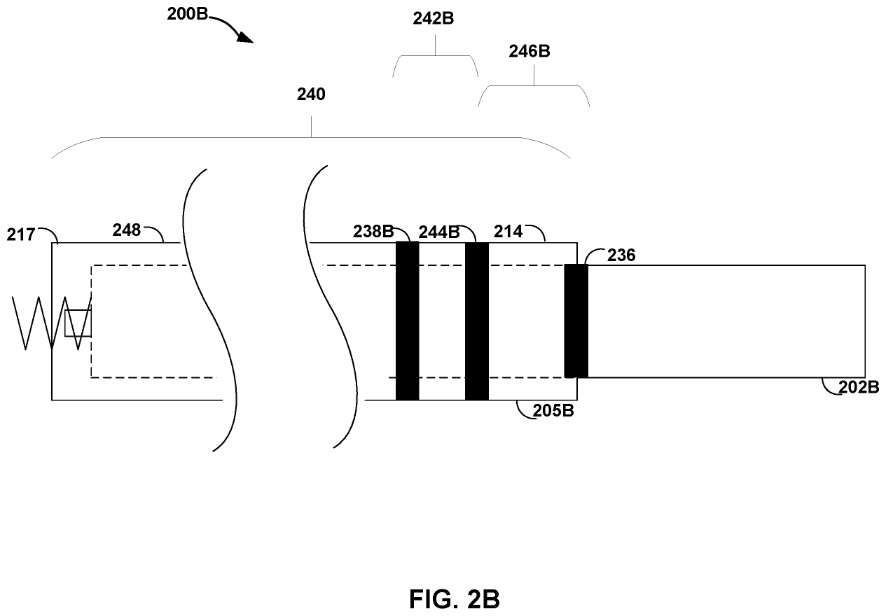

FIG. 2A is a conceptual drawing illustrating an example implant tool system 200A (“system 200A”). System 200A may be substantially similar to system 100 shown in FIG. 1, with the exception of any differences described herein. As shown in FIG. 2, system 200A includes an implantable medical lead 202A (“lead 202A”) and a delivery catheter 205A (“catheter 205A”) that extends from a proximal end 214 (“delivery catheter proximal end 214”) to a distal end 217 (“delivery catheter distal end 217”). Lead 202A may be substantially similar to lead 102, with the exception of any differences described herein. Catheter 205A may be substantially similar to catheter 105, with the exception of any differences described herein.

Lead 202A may include a fixation mechanism 232 to prevent lead 202A from shifting or moving (e.g., in a proximal direction). A fixation mechanism 232 configured to engage tissue may be attached to a distal end 212 of lead 202A (“lead distal end 212”). Fixation mechanism 232 may include, for example, one or more elongated tines such as fixation tines configured to substantially maintain an orientation of lead 202A with respect to implant point 106. Fixation mechanism 232 may include fixation tines of any shape, including helically-shaped fixation tines.

Fixation mechanism 232 may include a conductor, such as an electrically conductive material, having a non-conductive coating, such as polytetrafluoroethylene (PTFE), a portion (e.g., a distal end of fixation mechanism 232) of the conductive material being exposed to the tissue in which fixation mechanism 232 is embedded upon implantation of lead 202A. Electronic circuitry connected to lead 202 may be configured to generate and deliver electrical pulse therapy to the tissue proximate to fixation mechanism 232 via an electrode 234. In some examples, electrode 234 may be formed by a portion of fixation mechanism 232.

In some examples, lead 202A may include electrode 234 on lead distal end 212. That is, lead 202A may carry and/or mechanically support electrode 234. Electrode 234 may be electrically coupled to electronic circuitry via lead 202A. Electrode 234 may be configured to receive stimulation signals from lead 202A for delivery to tissue, as well as allow sensing circuitry of an implanted device to sense electrical signals from heart 108 via lead 202A. In some examples, lead 202A may be attached to tissue in the vicinity of implant point 106, thereby placing electrode 234 in contact with the tissue such that electrode 234 may deliver stimulation signals to tissue.

In examples, electrode 234 may be a shallow electrode configured for sensing and delivery of therapy signals to tissue in a substantially non-invasive manner. In other examples, electrode 234 may be a deep electrode configured to penetrate the tissues for sensing and delivery of therapy signals. Although electrode 234 is illustrated in FIG. 2A as a single electrode, it should be understood that other electrode configurations are contemplated, such as electrode configurations including two or more electrodes and/or in other locations of lead 202A.

In accordance with techniques described herein, system 200A may include a first visual marker 236 and a second visual marker 238A. First visual marker 236 and second visual marker 238A may be substantially similar to visual marker 118 except for any differences described herein. First visual marker 236 may be positioned on a body 220 of lead 202A (“lead body 220”). First visual marker 236 may be proximal lead distal end 212 by a first distance indicative of a minimum length of lead 202A to introduce into the body of patient 104 such that lead distal end 212 abuts implant point 206. Second visual marker 238A may be positioned on lead body 220. For instance, second visual marker 238A may be positioned on lead body 220 such that second visual marker 238A is proximal lead distal end 212 by a second lead distance indicative of an additional length of lead 202A to introduce into the body of patient 104 (e.g., to achieve desirable lead slack).

First visual marker 236 may be positioned such that alignment of first visual marker 236 with a reference point, such as delivery catheter proximal end 214, indicates that a minimum length 240 of lead 202 (“minimum lead length 240”) has been introduced into the patient's body. Thus, minimum lead length 240 may correspond to the first distance (which in turn may correspond to length 124 of FIG. 1B). In general, minimum lead length 240 may vary a relatively large amount (e.g., 10-50 centimeters (cm)) based on the patient. For example, minimum lead length 240 may vary based on factors including, but not limited to, body mass index (BMI), height, vasculature tortuosity, chosen access vessel, heart size, and implant point 106.

Minimum lead length 240 may be based on one or more factors, such as body mass index (BMI), height, vasculature tortuosity, chosen access vessel, heart size, and/or implant point 106. The relationship between minimum lead length 240 and the one or more factors may be provided as a formula. The relationship may be used to calculate an appropriate range of minimum lead length 24 based on the factors. The relationship may be determined in any appropriate manner. For example, a population of successful implant recipients may have both an anatomical dimension and minimum lead length 24 measured. In addition, various clinical or research studies may be used to determine the appropriate relationship. The relationship may be organized and presented in a suitable form (e.g., a table) to help a clinician determine minimum lead length 240. For example, a clinician may reference a table indicating minimum lead length 240 for a given BMI, height, vasculature tortuosity, chosen access vessel, heart size, and/or implant point 106. At least some of these anatomical measurements may be determined based on ultrasound images.

Second visual marker 238A may be positioned such that alignment of second visual marker 238A with a reference point (e.g., delivery catheter proximal end 214, access point 116, etc.) indicates that an additional length 242A of lead 202 (“additional lead length 242A”) has been further introduced into the patient's body. Additional length 242A may correspond to the second lead distance (which in turn may correspond to length 130 of FIG. 1B). As such, additional length 242 may be determined in a similar manner as length 130. In general, additional lead length 242A may vary a relatively small amount (e.g., 1-5 cm) based on patient 104. For example, additional lead length 242A may vary based on factors such as heart size, chest volume, and implant point 106.

In some examples, system 200A may include a third visual marker 244A positioned on lead 202A. Third visual marker 244A may be substantially similar to visual marker 118 except for any differences described herein. Third visual marker 244A may be proximal lead distal end 212 by a third lead distance indicative of a length of delivery catheter 205A to withdraw from the patient's body during introduction of lead 202A into the patient's body. Withdrawing delivery catheter 205A may be necessary to introduce additional length of lead 202A (e.g., additional length 242A). For instance, introducing additional length 242A without withdrawing catheter 205A may require a considerable amount of force (e.g., because both lead 202A and catheter 205A may need to flex), which may cause dislodgement of lead 202A and/or injury to patient 104. Third visual marker 244A may be positioned such that alignment of third visual marker 244A with delivery catheter proximal end 214 indicates that an appropriate length 246A of catheter 205 (“delivery catheter withdrawal length 246A”) has been withdrawn. Delivery catheter withdrawal length 246A may correspond to the third lead distance.

FIG. 2B is a conceptual diagram illustrating an example implant tool system 200B (“system 200B”). System 200B may be substantially similar to system 200A shown in FIG. 2A, with the exception of any differences described herein. As shown in FIG. 2B, system 200B includes an implantable medical lead 202B (“lead 202B”) and a delivery catheter 205B (“catheter 205B”). Lead 202B may be substantially similar to lead 202A, with the exception of any differences described herein. Catheter 205B may be substantially similar to catheter 205A, with the exception of any differences described herein.

Like system 200A, system 200B may include two or more visual markers. For instance, as shown in FIG. 2B, system 200B includes a first visual marker 236, a second visual marker 238B, and a third visual marker 244B, each of which may be substantially similar to visual marker 118. In the example of FIG. 2B, second visual marker 238B may be positioned on a body 248 of catheter 205 (“catheter body 248”). For instance, second visual marker 238B may be positioned on catheter body 248 such that second visual marker 238B is proximal catheter distal end 217 by a second catheter distance indicative of an additional length of lead 202B to introduce into the body of patient 104 (e.g., to achieve desirable lead slack). second visual marker 238B may be positioned on a body 248 of catheter 205 (“catheter body 248”). Third visual marker 244B may be positioned on catheter body 248 such that third visual marker 238B is proximal catheter distal end 217 by a third catheter distance indicative of a length of delivery catheter 205B to withdraw from the patient's body during introduction of lead 202B into the patient's body.

Second visual marker 238B may be positioned such that alignment of second visual marker 238B with a reference point (e.g., first visual marker 236 on lead 202) indicates that an additional length 242B of lead 202 (“additional lead length 242B”) has been further introduced into the patient's body. Additional length 242B may correspond to the second catheter distance (which in turn may correspond to length 130 of FIG. 1B). In general, additional lead length 242B may vary a relatively small amount (e.g., 1-5 cm) based on patient 104. For example, additional lead length 242B may vary based on factors such as heart size, chest volume, and implant point 106.

Third visual marker 244B may be positioned such that alignment of third visual marker 244B with a reference point (e.g., first visual marker 236) indicates that an appropriate length 246B of catheter 205 (“delivery catheter withdrawal length 246B”) has been withdrawn. Delivery catheter withdrawal length 246B may correspond to the third catheter distance.

Thus, the techniques of this disclosure may comprise a second visual marker positioned on either an implantable medical lead or a delivery catheter, and a third visual marker positioned on either the implantable medical lead or the delivery catheter. It should be understood that while FIGS. 2A-2B describe various examples of tool implant systems, other examples are contemplated. For instance, a system may include a second visual marker positioned on an implantable medical lead and a third visual marker positioned on a delivery catheter. In another example, a system may include a second visual marker positioned on a delivery catheter and a third visual marker positioned on an implantable medical lead. In any case, the positions of the visual markers and selection of reference points may be adjusted accordingly to perform the techniques of this disclosure.

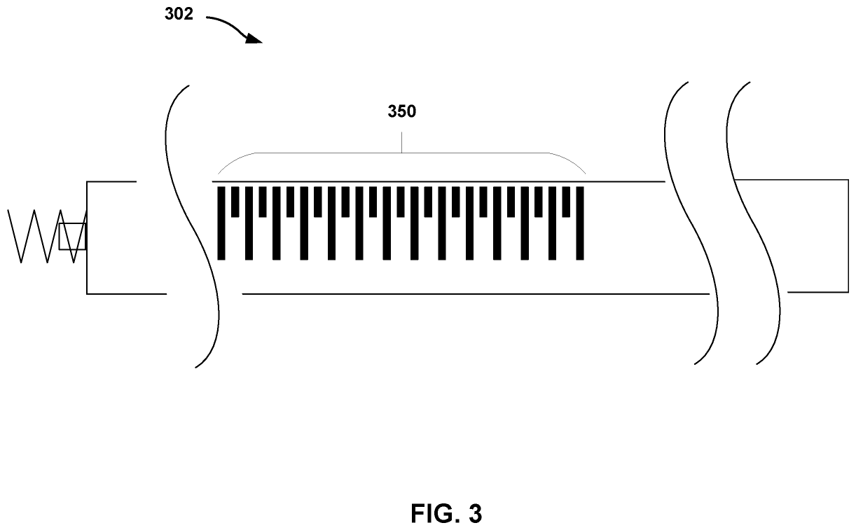

FIG. 3 is a conceptual drawing illustrating an example implantable medical lead 302 (“lead 302”), which is substantially similar to lead 102, lead 202A, and lead 202B. As shown in FIG. 3, lead 202C includes visual indicia 350 representing a series of measurement gradations. Visual indicia 350 may be used to position visual markers and/or measure movement of lead 302 along the implant path. For example, visual indicia 350 may represent ruler-like markers (e.g. transverse ridges or grooves), which may be seen and/or potentially felt. In other examples, visual indicia 350 may include numbering indicating units of length (e.g., inches, centimeters, etc.). Visual indicia 350 may be placed on the surface of and along at least a portion of a body 320 of lead 302 (“lead body 320”). In some examples, visual indicia 350 may not be integrated with lead 302 but instead associated with a sheath or a removable layer reversibly secured to lead 302.

During insertion of lead 302 into a delivery catheter (e.g., delivery catheter 105), the entire length of lead 302 moves such that visual markers (e.g., first visual marker 118, second visual marker 238A, visual indicia 250, etc.) associated with lead 302 likewise move. The clinician may determine the length of lead 302 inserted into the delivery catheter by visual or manual reference to the relative movement of visual indicia 350 to a reference point (e.g., delivery catheter proximal end 114). In this way, visual indicia 350 may help the clinician determine whether a sufficient amount of lead 302 has been introduced into patient's body.

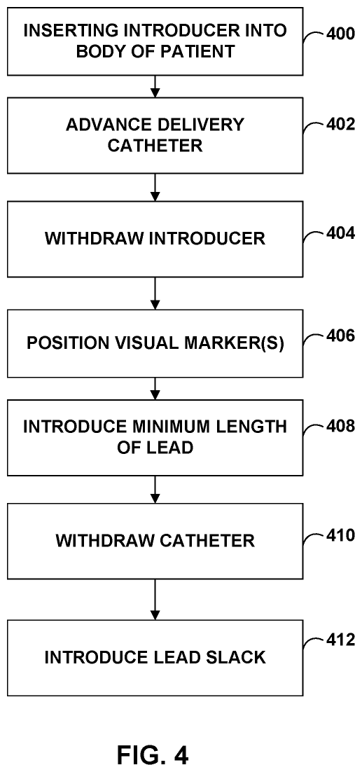

FIG. 4 is a flow diagram of an example technique for delivering an appropriate length of lead into a patient's body. Although FIG. 4 is discussed primarily in the context of system 100 of FIGS. 1A and 1B, it should be understood that the method of FIG. 4 may be applied to other examples of implant tool systems as described herein.

A clinician (or other medical practitioner) may insert introducer 122 into patient's body (400). For example, the clinician may insert introducer 122 through a lumen of catheter 105 and access point 106. The clinician may advance introducer 122 along an implantation path until introducer distal end 126 abuts (e.g., is proximate to) implant point 106. In some examples, advancement of introducer 122 (and/or other components of system 100) may be guided by ultrasound. By using ultrasound, a clinician may avoid or limit the usage of fluoroscopic observation, which may increase the safety of the implantation procedure.

The clinician may advance catheter 105 into the patient's body (402). For example, the clinician may translate catheter 105 relative to introducer 122 until delivery catheter distal end 117 abuts or is otherwise near to implant point 106. The clinician may withdraw introducer 122 (404). The clinician may observe the length of introducer 122 inserted into the patient's body before, during, and/or after withdrawing introducer 122. The clinician may position visual marker 118 (406). For instance, the clinician may align introducer distal end 126 with lead distal end 112 and use the length 124 to determine length 128.

The clinician may introduce the minimum length of lead 102 into the patient's body (408). In some examples, the clinician may use his sense of touch to determine when lead distal end 112 abuts implant point 106, which indicates that the minimum length of lead 102 has been introduced into the patient's body. The clinician may withdraw some length of catheter 105 (e.g., to enable introduction of lead slack without risking dislodgment of lead 102) (410). The clinician may fixate lead 102 and further advance lead 102 until visual marker 118 has aligned with a reference point (e.g., access point 106), indicating that an adequate amount of lead slack has been introduced (412).

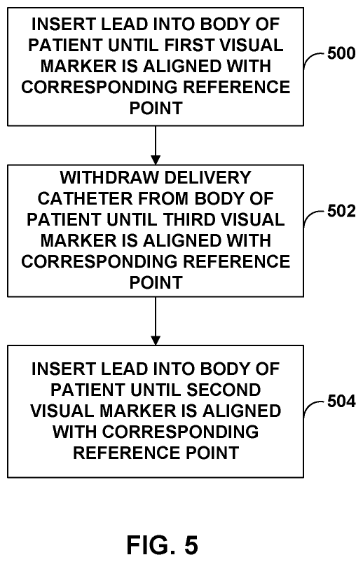

FIG. 5 is a flow diagram of an example technique for delivering an appropriate length of lead into a patient's body. Although FIG. 5 is discussed primarily in the context of system 200A of FIG. 2A, it should be understood that the method of FIG. 4 may be applied to other examples of implant tool systems as described herein.

A clinician may insert lead 202A into the patient's body until first visual marker 236 is aligned with a reference point, such as delivery catheter proximal end 214 (500). The clinician may position first visual marker 236 based on factors such as such as BMI, height, implant point 106, etc. Alignment of first visual marker 236 and the corresponding reference point may indicate that minimum lead length 240 has been introduced into the patient's body.

The clinician may withdraw catheter 205 until third visual marker 244A aligns with a corresponding reference point, such as delivery catheter proximal end 214 (502). Third visual marker 244A may be positioned such that alignment of third visual marker 244A with delivery catheter proximal end 214 indicates that delivery catheter withdrawal length 246A has been withdrawn.

The clinician may insert lead 202A into the patient's body until second visual marker 238A is aligned with a reference point, such as delivery catheter proximal end 214 (500). The clinician may position second visual marker 238A based on factors such as such as heart size, chest volume, implant point 106, etc. Alignment of second visual marker 238A and the corresponding reference point may indicate that additional lead length has been introduced into the patient's body.

This disclosure includes various examples, such as the following examples.

Example 1: An implant tool system includes an implantable medical lead configured to be delivered through an access point on a body of a patient; a delivery catheter configured to deliver the implantable medical lead along an implantation path in the body of the patient; a first visual marker, associated with the implantable medical lead, indicating a minimum length of lead to introduce into the body of a patient for delivering the medical lead through the access point and to a distal end of the catheter via the implant path; and a second visual marker, associated with the implantable medical lead or the delivery catheter, indicating an additional length of lead to introduce into the body of the patient.

Example 2: The implant tool system of example 1, further including a third visual marker, associated with the implantable medical lead or the delivery catheter, indicating a length to withdraw the delivery catheter from the body of the patient after fixation of the implantable medical lead to a tissue of the patient and before slitting the delivery catheter.

Example 3: The implant tool system of example 1 or 2, wherein the additional length is selected to provide lead slack to reduce the likelihood of one or more of dislodgment of the medical lead from the implant point or damage to a tissue of the heart.

Example 4: The implant tool system of any of examples 1-3, wherein one or more of the first visual marker or the second visual marker are movable relative to one or more of the implantable medical lead or the delivery catheter.

Example 5: The implant tool system of any of examples 1-4, wherein one or more of the first visual marker or the second visual marker are integrated into one or more of the implantable medical lead or the delivery catheter.

Example 6: The implant tool system of any of examples 1-5, wherein the second visual marker is associated with the implantable medical lead.

Example 7: The implant tool system of any of examples 1-6, wherein one or more of the implantable medical lead or the delivery catheter further includes visible indicia representing a series of measurement gradations.

Example 8: The implant tool system of any of examples 1-7, wherein the implantable medical lead is configured to be delivered through the access point on the body of the patient to an implant point in a heart of the patient.

Example 9: A method of using an implant tool system for delivering an implantable medical lead through an access point on a body of a patient along an implantation path in the body of the patient via a delivery catheter includes inserting, into the body of the patient, the implantable medical lead to a first visual marker, associated with the implantable medical lead, indicating a minimum length of lead to introduce into the body of a patient for delivering the implantable medical lead through the access point and to a distal end of the catheter via the implant path; and inserting, into the body of the patient, the implantable medical lead to a second visual marker, associated with the implantable medical lead or the delivery catheter, indicating an additional length of lead to introduce into the body of the patient.

Example 10: The method of example 9, further including withdrawing, from the body of the patient, the delivery catheter to a third visual marker, associated with one or more of the implantable medical lead or the delivery catheter, indicating a length to withdraw the delivery catheter from the body of the patient after fixation of the implantable medical lead to the implant point and before slitting the delivery catheter.

Example 11: The method of example 9 or 10, wherein the additional length is selected to provide lead slack to reduce the likelihood of one or more of dislodgment of the medical lead from the implant point or damage to a tissue of the heart.

Example 12: The method of any of examples 9-11, wherein one or more of the first visual marker and the second visual marker are movable relative to one or more of the implantable medical lead or the delivery catheter.

Example 13: The method of any of examples 9-12, wherein one or more of the first visual marker and the second visual marker are integrated into one or more of the implantable medical lead or the delivery catheter.

Example 14: The method of any of examples 9-13, wherein the second visual marker is associated with the implantable medical lead.

Example 15: The method of any of examples 9-14, wherein one or more of the implantable medical lead or the delivery catheter further includes visible indicia representing a series of measurement gradations.

Example 16: The method of any of examples 9-15, wherein the implantable medical lead is configured to be delivered through the access point on the body of the patient to an implant point in a heart of the patient.

Example 17: The method of any of examples 9-16, further including positioning the first marker relative to the implantable medical lead based on one or more of a body mass index of the patient, height of the patient, vasculature tortuosity of the patient, an access vessel of the patient, a location along the access vessel of the patient, a heart size of the patient, or the implant point.

Example 18: The method of any of examples 9-17, further including positioning the first marker relative to the implantable medical lead based on a length of the delivery catheter from a proximal end of the delivery catheter to a distal end of the delivery catheter.

Example 19: The method of any of examples 9-18, further including positioning the second marker relative to the implantable medical lead based on one or more of a body mass index of the patient, height of the patient, vasculature tortuosity of the patient, an access vessel of the patient, a location along the access vessel of the patient, a heart size of the patient, or the implant point.

Example 20: The method of any of examples 9-19, further including positioning the third marker relative to one or more of the implantable medical lead or the delivery catheter based on one or more of the heart size of the patient, a chest volume of the patient, or the implant point.

Example 21: The method of any of examples 9-20, wherein inserting the implantable medical lead to the second visual marker as withdrawing the delivery catheter to the third visual marker is performed at the same time.

Example 22: The method of any of examples 9-21, wherein inserting the implantable medical lead to the first marker indicates that the distal end of the implantable medical lead is at a distal end of the delivery catheter.

Various aspects of the disclosure have been described. These and other aspects are within the scope of the following claims.

What is claimed is:

1. An implant tool system comprising:
an implantable medical lead configured to be delivered through an access point on a body of a patient;
a delivery catheter configured to deliver the implantable medical lead along an implantation path in the body of the patient; and
a visual marker positioned:
on a body of the implantable medical lead,
and proximal a distal end of the implantable medical lead by a distance indicative of a length of lead to introduce into the body of the patient.

2. The implant tool system of claim 1, wherein the visual marker is positioned such that introduction of the length of lead into the body of the patient provides lead slack that reduces the likelihood of one or more of dislodgment of the implantable medical lead from the implant point or damage to a tissue of the patient.

3. The implant tool system of claim 1, wherein the visual marker is configured to be integrated into the body of the implantable medical lead or reversibly secured to the body of the implantable medical lead.

4. The implant tool system of claim 1, wherein one or more of the implantable medical lead or the delivery catheter further comprises visible indicia representing a series of measurement gradations.

5. A method of using an implant tool system for delivering an implantable medical lead through an access point on a body of a patient, the method comprising:
inserting, into the body of the patient, an introducer;
advancing the introducer along an implantation path in the body of the patient until a distal end of the introducer abuts an implant point;
positioning, based on the length of the introducer inserted into the body of the patient when the distal end of the introducer abuts the implant point, a visual marker on a body of the implantable medical lead, wherein the visual marker is positioned proximal a distal end of the implantable medical lead by a distance indicative of a length of lead to introduce into the body of the patient.

6. The method of claim 5, wherein the visual marker is positioned such that introduction of the length of lead into the body of the patient provides lead slack that reduces the likelihood of one or more of dislodgment of the implantable medical lead from the implant point or damage to a tissue of the patient.

7. The method of claim 5, wherein the visual marker is configured to be integrated into the body of the implantable medical lead or reversibly secured to the body of the implantable medical lead.

8. The method of claim 5, wherein one or more of the implantable medical lead or the delivery catheter further comprises visible indicia representing a series of measurement gradations.

9. The method of claim 5, wherein the implantable medical lead is configured to be delivered through the access point on the body of the patient to an implant point in a heart of the patient.

10. The method of claim 5, wherein positioning the visual marker on the body of the implantable medical device is further based on one or more of a body mass index of the patient, height of the patient, vasculature tortuosity of the patient, an access vessel of the patient, a location along the access vessel of the patient, a heart size of the patient, or the implant point.

11. An implant tool system comprising:
an implantable medical lead configured to be delivered through an access point on a body of a patient;
a delivery catheter configured to deliver the implantable medical lead along an implantation path in the body of the patient;
a first visual marker positioned:
on a body of the implantable medical lead, and
proximal a distal end of the implantable medical lead by a first distance indicative of a minimum length of lead to introduce into the body of a patient such that the distal end of the implantable medical lead abuts an implant point; and
a second visual marker positioned:
on the body of the implantable medical lead and proximal the distal end of the implantable medical lead by a second lead distance indicative of an additional length of lead to introduce into the body of the patient, or
on the body of the delivery catheter and proximal a distal end of the delivery catheter by a second catheter distance indicative of the additional length of lead to introduce into the body of the patient.

12. The implant tool system of claim 11, further comprising:
a third visual marker positioned:
on the body of the implantable medical lead and proximal the distal end of the implantable medical lead by a third lead distance indicative of a length to withdraw the delivery catheter from the body of the patient after fixation of the implantable medical lead to a tissue of the patient and before slitting the delivery catheter, or
on the body of the delivery catheter and proximal the distal end of the delivery catheter by a third catheter distance indicative of the length to withdraw the delivery catheter from the body of the patient after fixation of the implantable medical lead to the tissue of the patient and before slitting the delivery catheter.

13. The implant tool system of claim 11, wherein the second visual marker is positioned such that introduction of the length of lead into the body of the patient provides lead slack that reduces the likelihood of one or more of dislodgment of the implantable medical lead from the implant point or damage to a tissue of patient heart.

14. The implant tool system of claim 11, wherein the second visual marker is configured to be integrated into the body of the implantable medical lead or reversibly secured to the body of the implantable medical lead.

15. The implant tool system of claim 11, wherein the second visual marker is configured to be integrated into the body of the delivery catheter or reversibly secured to the body of the delivery catheter.

16. The implant tool system of claim 11, wherein the implant point is in a heart of the patient.

17. The implant tool system of claim 11, wherein the position of the first visual marker is based on one or more of a body mass index of the patient, height of the patient, vasculature tortuosity of the patient, an access vessel of the patient, a location along the access vessel of the patient, a heart size of the patient, or the implant point.

18. The implant tool system of claim 11, wherein the position of the first visual marker is based on a length of the delivery catheter from a proximal end of the delivery catheter to a distal end of the delivery catheter.

19. The method of claim 9, wherein the position of the second visual marker is based on one or more of a body mass index of the patient, height of the patient, vasculature tortuosity of the patient, an access vessel of the patient, a location along the access vessel of the patient, a heart size of the patient, or the implant point.

20. The method of claim 12, wherein the position of the third marker is based on one or more of the heart size of the patient, a chest volume of the patient, or the implant point.
